(19) United States
(12) Patent Application Publication
(10) Pub. No.: US 2025/0331773 A1
(43) Pub. Date: Oct. 30, 2025

(54) PH SENSOR WITH PANI-COATED CONDUCTIVE THREADS, AND METHOD OF MANUFACTURE

(71) Applicant: FIBRA INC., Vaughan (CA)

(72) Inventors: Parnian MAJD, Vaughan (CA); Judith Castillo RODRIGUEZ, Scarborough (CA)

(21) Appl. No.: 19/191,745

(22) Filed: Apr. 28, 2025

Related U.S. Application Data
(60) Provisional application No. 63/639,248, filed on Apr. 26, 2024.

(51) Int. Cl. A61B 5/00; A61B 5/145; C09D 139/00; D01D 5/00; D02G 3/40; D02G 3/44

(52) U.S. Cl. CPC A61B 5/6804; A61B 5/14507; A61B 5/14539; A61B 5/43; C09D 139/00; D01D 5/0084; D02G 3/404; D02G 3/441; A61B 2562/046; D10B 2101/20

(57) ABSTRACT

A method of manufacturing a textile-based pH sensor is provided. Conductive threads are coated with polyaniline by either electrospinning to form a fibrous mat or by depositing a film from a polymeric solution comprising 1 to 10% polyaniline and 10 to 20% polymer matrix. The coated threads are integrated into a textile and spaced apart. A control unit is connected to the threads to apply a test signal, detect a feedback signal when a biological liquid connects the threads, and determine the pH based on signal comparison. The fibrous or film polyaniline coatings enhance sensitivity, flexibility, and durability for wearable applications.

### CROSS-REFERENCE TO RELATED APPLICATION

This application claims the benefit of U.S. Provisional Patent Application No. 63/639,248 entitled “PH SENSOR FOR WEARABLE DEVICE”, filed Apr. 26, 2024, the entire contents of which are incorporated herein by reference.

### FIELD

The present specification is directed to wearable devices, and in particular, a pH sensor comprising conductive threads for integration in a wearable device.

### BACKGROUND

Wearable devices, or “smart clothes,” often use screen-printed sensors integrated into fabrics to monitor physiological parameters such as heart rate and temperature in real time. These systems must balance comfort with sensor flexibility, durability for repeated wear and washing, and scalability for cost-effective production. Consistent performance across manufacturing batches is also essential for maintaining reliability and user confidence.

Conventional Ag/AgCl electrodes, while effective for testing pH in a laboratory setting, are not washable and lack the mechanical flexibility needed for textile integration. The AgCl layer is brittle and prone to cracking or delamination under flexing, and its stability depends on maintaining a fixed chloride concentration, which is difficult to control in wearable environments.

### SUMMARY

The specification provides a pH sensor that addresses the challenges of flexibility, durability, and manufacturability in wearable applications. Conductive threads are coated with polyaniline (PANI) improves electrical performance while allowing the threads to move naturally with the fabric. These threads can be incorporated into textiles and electrically coupled to control circuitry that enables accurate, real-time pH measurement of biological fluids.

An aspect of the specification provides a pH sensor for characterizing biological liquids. The sensor includes a plurality of conductive threads disposed on a textile. The threads include at least a first conductive thread and a second conductive thread spaced apart from each other. The threads include a fibrous polyaniline coating. A control unit is electrically connected to the conductive threads. The control unit is configured to apply a test signal to the first conductive thread, to record a feedback signal in the second conductive thread in response to a biological liquid electrically connecting the first and second conductive threads, to compare the test signal to the feedback signal, and to determine a pH of the biological liquid based on the comparison.

In one example, the plurality of conductive threads include one or more of silver, copper, gold, aluminum, iron, steel, brass, graphite, conductive polymers, carbon nanotubes, and alloys thereof.

In one example, the fibrous polyaniline coating includes a fibrous mat.

In one example, the conductive threads are stitched into the textile.

In one example, the conductive threads are arranged on the textile as interdigitated electrodes.

In one example, the conductive threads are arranged substantially parallel to each other on the textile.

In one example, the control unit is further configured to detect whether the feedback signal is transmitted by the second conductive thread and to determine that no biological liquid is present on the textile if the signal is not transmitted.

A further aspect of the specification provides a method of manufacturing a pH sensor. Polyaniline is suspended in a polymeric solution, and this solution is loaded into a syringe connected to a spinneret. A voltage is applied to the spinneret to induce an electrospinning effect. The polymeric solution is ejected through the spinneret to form polyaniline fibers, which are deposited onto a conductive thread to form a fibrous polyaniline coating on the thread.

In one example, the method includes controlling one or more of the voltage, spinneret diameter, solution feed rate, and the distance between the spinneret and the conductive thread to optimize a characteristic of the fibrous polyaniline coating.

In one example, the method includes selecting a particle size of the polyaniline to optimize the suspension.

In one example, the conductive threads include one or more of silver, copper, gold, aluminum, iron, steel, brass, graphite, conductive polymers, carbon nanotubes, and alloys thereof.

In some examples, two of the conductive threads are stitched into a textile or garment. The two conductive threads may be arranged to form an interdigitated electrode.

In some examples, the method includes connecting the two conductive threads to a control unit which is configured to measure the pH of a biological liquid contacting the two conductive threads.

An aspect of the specification provides a method of manufacturing a pH sensor. The method includes suspending polyaniline in a polymeric solution comprising about 1 to 10% polyaniline and about 10 to 20% of a polymer matrix, and depositing a film comprising the polyaniline onto a conductive thread.

In one example, depositing the polyaniline film onto the conductive thread is performed by drop casting or doctor blading.

In one example, the method further includes selecting a particle size of the polyaniline to optimize the suspension.

In one example, the conductive threads include one or more of silver, copper, gold, aluminum, iron, steel, brass, graphite, conductive polymers, carbon nanotubes, and alloys thereof.

In one example, the method further includes stitching two conductive threads into a textile and connecting the two conductive threads to a control unit configured to measure the pH of a biological liquid contacting the two conductive threads.

In one example, stitching the two conductive threads into the textile further comprises arranging the two conductive threads to form an interdigitated electrode.

A further aspect of the specification provides a fertility monitoring system. The system includes a pH sensor for characterizing a biological liquid. The sensor includes a plurality of conductive threads disposed on a textile, including a first conductive thread and a second conductive thread spaced apart from each other. The threads include a fibrous polyaniline coating. The system also includes a control unit electrically connected to the conductive threads. The control unit is configured to apply a test signal to the first conductive thread and to record a feedback signal in the second conductive thread in response to a biological liquid electrically connecting the threads. A computing device is configured to receive the test and feedback signals, to compare the signals, and to determine a pH of the biological liquid based on the comparison.

In one example, the control unit is further configured to detect whether the feedback signal is transmitted by the second conductive thread and to determine that no biological liquid is present on the textile if the signal is not transmitted.

In one example, the computing device is further configured to compare the pH of the biological liquid to reference data and to determine a reproductive status of a user based on the comparison between the pH and the reference data.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Embodiments are described with reference to the following figures.

FIG. 1 is a front elevation view of a wearable device including a pH sensor and sensing element, according to one embodiment.

FIG. 2 is a block diagram of the pH sensor of FIG. 1, according to one embodiment.

FIG. 3 is a block diagram of a fertility monitoring system including the pH sensor of FIG. 1.

FIG. 4A is a top elevation view of the sensing element of FIG. 1, according to one embodiment.

FIG. 4B is a top elevation view the sensing element of FIG. 1, according to another embodiment.

FIG. 5A is a top elevation view of the sensing element of FIG. 1, according to a further embodiment.

FIG. 5B is a top elevation view of the sensing element of FIG. 1, according to a yet further embodiment.

FIG. 6 is a block diagram of a method for detecting the pH of a biological liquid using the sensing element of FIG. 1, according to one embodiment.

FIG. 7A is a diagram of an electrospinning apparatus 700 for manufacturing the pH sensor of FIG. 1.

FIG. 7B is a block diagram of a method for manufacturing the pH sensor of FIG. 1 using the apparatus of FIG. 7B.

### DETAILED DESCRIPTION

The present specification provides a pH sensor comprising polyaniline-coated conductive threads for detecting the pH of a biological liquid. In the embodiments described herein, the pH sensor is adapted for use in a wearable device, however the pH sensor is not particularly limited and may be applied to any suitable textile.

The following definitions are used herein:

“Polyaniline” or “PANI” herein refers to a conductive polymer comprising aniline (also known as benzenamine) monomers.

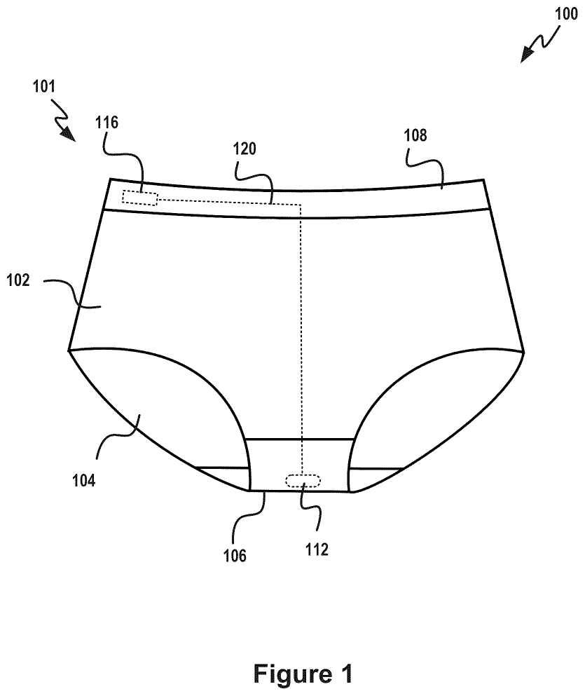

FIG. 1 is a front elevation view of a wearable device 100 including a pH sensor 101 according to one embodiment. In the example shown in FIG. 1, the wearable device 100 comprises underwear, however the wearable device 100 is not particularly limited. In other embodiments, the wearable device 100 comprises an undershirt, bra, headpiece, leggings, swimwear, shapewear, shirt, sock, wristband, or any suitable garment. The wearable device 100 generally comprises one or more textile portions to be worn on the user's body. In this example, the textile portions comprise a front portion 102, a rear portion 104, a gusset 106 and a waistband 108, however other configurations are contemplated. One or more of the textile portions may comprise a plurality of textile layers.

The textile portions may comprise any suitable woven or non-woven fabric. In examples where the textile portions comprise a woven fabric, the textile may include but is not limited to cotton, silk, linen, wool, polyester, nylon, rayon, modal, and a combination thereof. The textile may be selected to optimize the distribution and drying time of liquids contacting the textile. The drying time for absorbent fabrics like cotton is generally faster than the drying time for non-absorbent fabrics like nylon. The distribution of liquids is generally better on absorbent fabrics as opposed to non-absorbent fabrics. In some examples, the textile is selected to achieve a distribution time of about 5 to 10 seconds. In specific non-limiting examples, the textile comprises a fabric blend of cotton and polyester, and in particular examples about 10% polyester and about 90% cotton.

The pH sensor 101 comprises a sensing element 112 for detecting the acidity a biological liquid and a control unit 116 for receiving signals from the sensing element 112 via one or more connectors 120. The sensing element 112 is disposed on one of the textile portions of the wearable device 100. In the example shown in FIG. 1, the sensing element 112 is disposed on the gusset 106, however the sensing element 112 is not particularly limited. In other embodiments, the sensing element 112 is disposed on the rear, front, or waistband of the wearable device 100. Generally, the sensing element 112 is positioned to capture biological liquids secreted by the user.

The control unit 116 is configured to apply a test signal to the biological liquid via the sensing element 112 and receive a feedback signal indicative of the acidity of the biological liquid. The control unit 116 is configured to transmit the test signal to the sensing element 112 via the connector 120. The sensing element 112 is configured to transmit the feedback signal to the control unit 116 via the connector 120.

The connector 120 electrically connects the sensing element 112 to the control unit 116. The connector 120 may be disposed between two layers of textile, disposed on the surface of a layer of textile, knitted into the textile, stitched into the textile, or woven into the textile of the wearable device 100. In specific embodiments, the connector 120 comprises a conductive thread that is incorporated into the textile portion of the wearable device 100. The connector 120 may comprise any suitable conductive material such as stainless steel. A coating may cover the connector 120 to protect the connector from oxidization.

The control unit 116 is preferably located in the waistband 108 of the wearable device 100 but the control unit 116 is not particularly limited. The control unit 116 applies a test signal to the sensing element 112 and receives a feedback signal responsive to the test signal.

In some examples, the wearable device 100 does not include a control unit 116 and instead includes a wireless transmitter for transmitting the feedback signal wirelessly. Suitable examples of a wireless transmitter may include a Wi-Fi module, a Bluetooth™ module, radiofrequency identification (RFID) tag, the like, or combinations thereof.

In specific, non-limiting embodiments, the control unit 116 includes the Arduino™ UNO (Arduino: New York, United States) or the Arduino™ Nano 33 BLE (Arduino: New York, United States), however the control unit 116 is not particularly limited.

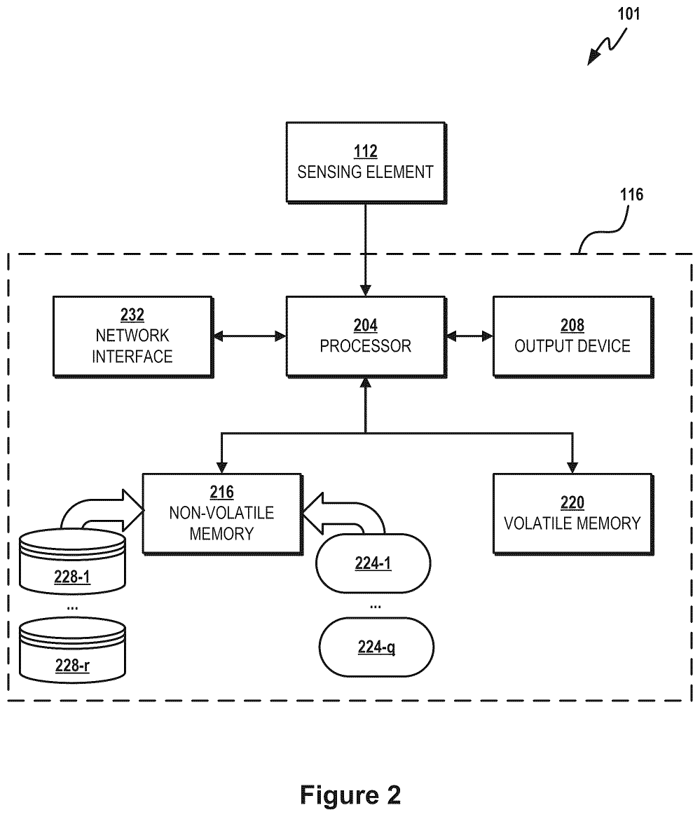

FIG. 2 in a block diagram of the pH sensor 101 showing control unit 116 in greater detail. The control unit 116 may comprise a processor 204 for receiving a feedback signal from sensing element 112 and processing said feedback signal to generate an output.

The processor 204 may be implemented as a plurality of processors or one or more multi-core processors. The processor 204 may be configured to execute different programing instructions responsive to the feedback received from the sensing element 112 and to control one or more output devices 208 to generate output on those devices.

To fulfill its programming functions, the processor 204 is configured to communicate with one or more memory units, including non-volatile memory 216 and volatile memory 220. Non-volatile memory 216 can be based on any persistent memory technology, such as an Erasable Electronic Programmable Read Only Memory (“EEPROM”), flash memory, solid-state hard disk (SSD), other type of hard-disk, or combinations of them. Non-volatile memory 216 may also be described as a non-transitory computer readable media. Also, more than one type of non-volatile memory 216 may be provided.

The volatile memory 220 is based on any random-access memory (RAM) technology. In specific, non-limiting examples, volatile memory 220 can be based on a Double Data Rate (DDR) Synchronous Dynamic Random-Access Memory (SDRAM). Other types of volatile memory 220 are contemplated.

The processor 204 also connects to a network 236 via a network interface 232. Suitable examples of network interfaces may include a Wi-Fi module, a Bluetooth™ module, a radio frequency identification (RFID) tag, the like, or a combination thereof.

Programming instructions in the form of applications 224 are typically maintained, persistently, in the non-volatile memory 216 and used by the processor 204 which reads from and writes to the volatile memory 220 during the execution of the applications 224. Various methods discussed herein can be coded as one or more applications 224. (Generically referred to herein as “application 224” or collectively as “applications 224”. This nomenclature is used elsewhere herein.)

One or more tables or databases 228 are maintained in non-volatile memory 216 for use by applications 224.

The control unit 116 may further include a potentiostat (not shown) for measuring the electrical potential of the biological liquid.

The control unit 116 further comprises a power source (not shown) for applying a test signal to the sensing element 112 and powering the control unit 116. The power source may be integrated with or connected to the control unit 116. The power source may include a battery, a power port, a self-charging power pack, a power generation unit, or a combination thereof. In examples where the power source is a battery, the battery may be a rechargeable or non-rechargeable battery. The battery may be removable or permanent. In embodiments that include a power port for receiving power from an external source, the power source may further include a battery, and the power port may be configured to charge said battery.

In specific examples, the power source comprises one or more lithium-ion batteries to power the pH sensor 101. The control unit 116 may be powered via a USB (universal serial bus) port which is connected to the power source. The accompanying batteries might be stored in a 3D-printed box and located on the wrist of the user ensuring comfort and safety while using the wearable device.

In specific, non-limiting embodiments, the power source includes the Panasonic™ Lumix Li-Ion Battery Pack (model no. DMW-BLF19). The 7.2V, 1860 mAh battery potentially works for up to 24 hours if the operating voltage of the control unit 116 is between 7 to 14 V. Other power sources such as fully self-charging power packs (FSPP).

In further non-limiting embodiments, the power source includes the Molex™ Thin-Film Battery (Mouser Electronics: Kitchener, Canada). the Molex™ Thin-Film Battery may be used to power the control unit 116 and the sensing element 112. The Molex™ battery has a shelf life of about two years and can operate in a humidity of about 20% to about 90% and in a temperature range of about −35° C. to about 50° C. It is a 3V battery with an initial internal resistance of about 90 ohms and a peak current (maximum) of about 8 to about 10 mA. It is bendable and small. It has a minimum bending radius of about 35.00 mm, a thickness of about 0.70 mm, and a width of about 36.00 mm.

In embodiments where the power source includes a power generation unit, the power source may comprise a thermoelectric generator, a solar cell, piezoelectric device, an electromagnetic generator, the like, or combinations thereof.

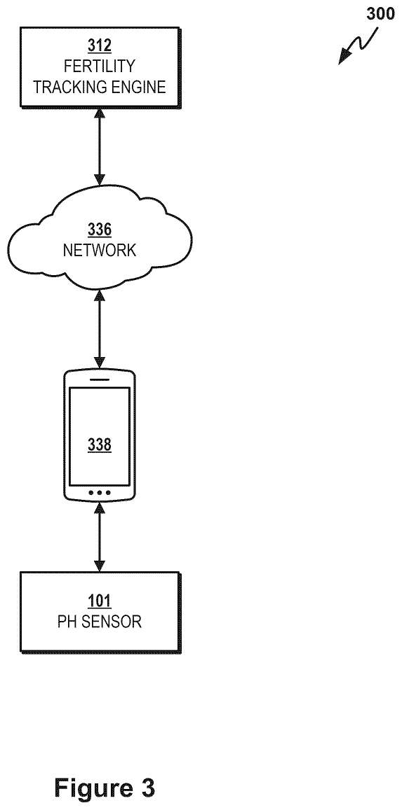

The network interface 232 can be used to connect a computing device, thereby obviating the need for one of more components of the control unit 116. FIG. 3 shows a fertility monitoring system 300 according to one embodiment in which the pH sensor 101 connects to a computing device 338.

The computing device 338 can be any type of human-machine interface for interacting with the pH sensor 101. For example, the computing device 338 may include a smartphone, a personal computer, a tablet computer, a smartwatch, a smart home system, or any other device that can be used to receive and send content. The computing device 338 can be operated by a user associated with a respective identifier that uniquely identifies the user accessing the computing device 338. The computing device 338 may comprise a processor for executing programming instructions in the form of applications. The computing device 238 may further include non-volatile memory. The computing device 238 may further include volatile memory. The computing device 338 may further include an output device. Any description of the processor 204 may apply to the processor of the computing device 338 and vice versa. Likewise, any description of the non-volatile memory 216 and volatile memory 220 may apply to the non-volatile and volatile memory of the computing device 338 and vice versa. Similarly, any description of the output device 208 may apply to the output of the computing device 338 and vice versa.

The computing device 338 may include a network interface for connecting to a fertility tracking engine 312 via a network 336. The fertility tracking engine 312 comprises volatile and non-volatile memory for storing fertility data associated with a unique identifier for identifying the user associated with the computing device 338. The fertility tracking engine 312 further includes a processor for executing programming instructions in the form of applications. Any description of the processor 204 may apply to the processor of the fertility tracking engine 312 and vice versa. Likewise, any description of the non-volatile memory 216 and volatile memory 220 may apply to the non-volatile and volatile memory of the fertility tracking engine 312 and vice versa.

Reference data may be stored in memory at the control unit 116, computing device 338, or fertility tracking engine 312. The reference data comprises feedback signals obtained from a plurality of test subjects using the pH sensor 101. The reference data may be associated with temporal, physiological, and demographic data. Temporal data may comprise a phase or a day within a reproductive cycle. Phases of the reproductive cycle include luteal phase, follicular phase, ovulation, fertile phase, proliferative phase, secretory phase, period, pregnancy, and the like. A day may be indicated as “Day 1 of 31” or the like. Physiological data may comprise a physiological indicator corresponding with the respective temporal data. In a specific example, the physiological data may comprise average body temperatures corresponding to days of the reproductive cycle. The demographic data may include age, weight, ethnicity, health status, disease state, and the like. The reference data may represent feedback data obtained for known biological fluids, including recorded times, electrical properties of the feedback signal, the number and placement of active conductive threads, and the gap distance between the conductive threads. A person of skill in the art will understand that the reference data may represent the average human reproductive cycle, and the biological liquids secreted during respective phases of the human reproductive cycle.

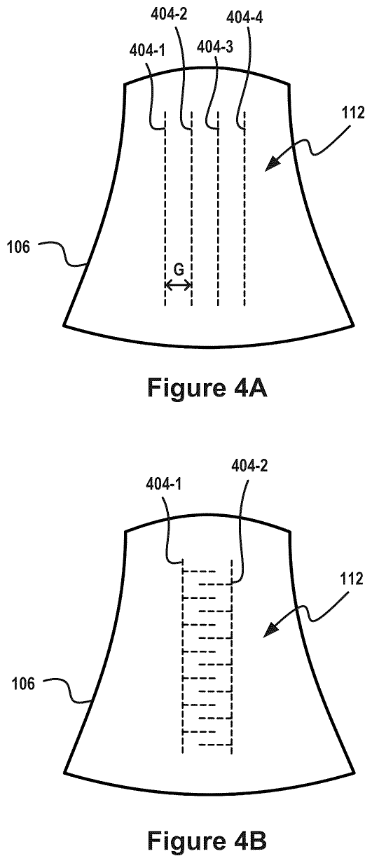

FIGS. 4A and 4B show the sensing element 112 in greater detail. In the embodiments shown in FIGS. 4A and 4B, the sensing element 112 is arranged on the gusset 106, however the sensing element 112 is not particularly limited. Generally, the sensing element 112 is configured to be woven or stitched onto the textile.

The sensing element 112 comprises a plurality of conductive threads 404-1, 404-2, 404-3, 404-4 (referred to herein generally as “conductive thread 404” or collectively as “conductive threads 404”) disposed on a textile. The sensing element 112 may include any suitable number of conductive threads 404. In some examples, the sensing element 112 includes two conductive threads 404. In some examples, the sensing element 112 includes four conductive threads 404. In some examples, the sensing element 112 includes six conductive threads 404. In some examples, the sensing element 112 includes ten conductive threads 404. In some examples, the sensing element 112 includes twenty conductive threads 404. In some examples, the sensing element 112 includes one hundred conductive threads 404. Generally, when the pH sensor 101 includes a high number of spaced apart conductive threads, it will be able to detect even small volumes of the biological liquid. In examples where the textile comprises a woven fabric, the conductive threads 404 may be integrated into the textile during the weaving of the textile. In other examples, the conductive threads 404 are stitched onto the textile. In specific non-limiting examples, the conductive threads 404 are stitched onto the textile using embroidery. In further non-limiting examples, the conductive threads 404 are disposed on the textile using adhesive.

The conductive threads 404 are connected to the control unit 116 via the connector 120. In some examples, each of the conductive threads 404 is connected to the control unit 116 by respective connectors 120.

The conductive threads 404 comprise any suitable natural or synthetic fiber including but not limited to silver, copper, gold, aluminum, iron, steel, brass, graphite, conductive polymers, carbon nanotubes, and the like. In specific, non-limiting examples, the conductive threads 404 comprise stainless steel yarn. The conductive threads 404 may be selected for stability, washability, ability to dry quickly, repeatability, durability, flexibility, biocompatibility, and antimicrobial properties. Specific non-limiting examples of conductive threads can be obtained from Mayata™, Shieldex™, VtechTextile™, Seeed Studio™, and other suitable suppliers.

The conductive threads 404 further comprise a polyaniline (PANI) coating. Generally, the polyaniline comprises either a film or a fibrous coat.

In examples where the PANI coating is a film, the polyaniline is synthesized using the monomer aniline (Sigma Aldrich™) and ammonium persulphate (Sigma Aldrich™), as well as the doping material Dodecyl Benzene Sulphonic Acid (TCI America™). The polyaniline is suspended in a polymeric solution comprising a polymer matrix. The polymer matrix may comprise polylactic acid (PLA), thermoplastic polyurethane (TPU), polyacrylonitrile (PAN), similar non-water-soluble polymers, or combinations thereof. The concentration of the polyaniline in the polymer matrix may be between about 1% and about 10% of the suspension by weight. The concentration of the polymer matrix may comprise between about 10% and about 20% of the suspension by weight. In some examples, the polymer matrix and the polyaniline may have a ratio of 1:1 in the suspension. The particle size of the polyaniline may be optimized for suspension in the polymeric matrix. The polyaniline may be further optimized for conductivity. The polyaniline may be deposited onto the conductive thread 404 as a film using electrochemical deposition or drop casting or doctor blading.

This approach employs a flexible, scalable fabrication method using thin-film deposition techniques such as drop casting and doctor blading. These methods are more cost-effective and simpler compared to the fiber weaving and integration techniques used in the past. Additionally, the method leverages a broader range of material combinations by incorporating non-water-soluble polymers like PLA, TPU, and PAN as the matrix for PANI particles. This provides greater versatility in optimizing the sensor's properties for specific applications, such as wearable electronics or environmental sensors, while offering enhanced durability and performance. In contrast, fiber-based sensors are more limited in material selection and scalability. This approach facilitates easier integration into a wide variety of substrates and devices, offering a more adaptable and commercially viable solution for diverse applications.

In other examples, the polyaniline coating comprises a fibrous coat. The conductive threads 404 further comprise a fibrous polyaniline (PANI) coating. In specific non-limiting embodiments, the polyaniline is synthesized using the monomer aniline (Sigma Aldrich™) and ammonium persulphate (Sigma Aldrich™), as well as the doping material Dodecyl Benzene Sulphonic Acid (TCI America™). The polyaniline may be suspended in a polymeric solution, suitable for electrochemical deposition or electrospinning. The particle size of the polyaniline may be optimized for suspension in the polymeric solution. The polyaniline may be further optimized for conductivity. The polyaniline may be deposited onto the conductive thread 404 as a film using electrochemical deposition or a fibrous mat using electrospinning. Since polyaniline has a crystalline structure which is only semi-flexible, the fibrous coating provides improved flexibility and durability as compared to the film coating. The fibrous form of polyaniline may also improve contact with the biological liquid.

The conductive threads 404 are spaced apart in the textile. In some examples, the conductive threads 404 are equidistant or approximately equidistant. In other examples, the gap distance G between adjacent conductive threads 404 vary, with some of the conductive threads 404 positioned closer and other positioned farther apart. The gap distance G between adjacent conductive threads 404 may be selected based on the desired sensitivity or properties of the biological liquid. The gap distance G between adjacent conductive threads may range from about 0.5 mm to about 100 mm. In specific examples, the gap distance G is about 0.5 mm. In specific examples, the gap distance G is about 1 mm. In specific examples, the gap distance G is about 2 mm. In specific examples, the gap distance G is about 4 mm. In specific examples, the gap distance G is about 6 mm. In specific examples, the gap distance G is about 8 mm. In specific examples, the gap distance G is about 10 mm. In specific examples, the gap distance G is about 15 mm. In specific examples, the gap distance G is about 10 mm. In specific examples, the gap distance G is about 20 mm.

It should be understood that the sensitivity of the pH sensor 101 is correlated with the number of the conductive threads 404 and the respective gap distances G. Generally, if the first and second conductive threads 404-1, 404-2 are positioned close together, the pH sensor 101 will be able to detect even small volumes of biological liquid. Furthermore, the precision will be correlated with the number of the conductive threads 404 included in the pH sensor 101.

Preferably, the conductive threads 404 are not in contact with each other, so that the conductive threads 404 are not electrically connected. Any number of configurations are contemplated for spacing the conductive threads 404. In FIG. 4A, the conductive threads are spaced apart and arranged substantially parallelly on the textile, however the conductive threads 404 are not particularly limited. In some embodiments, the conductive threads are aligned in straight lines, curved lines, zigzags, radial pattern, grid pattern, abstract shapes, concentric circles, scattered dots, or the like. In FIG. 4B, the first conductive thread 404-1 and the second conductive thread 404-2 are arranged on the textile as interdigitated electrodes (IDEs). The IDEs may improve the resolution of changes in the polyaniline. The interdigitated arrangement may also improve sensitivity, allowing the pH sensor 101 to detect even small amounts of biological liquids. This feature is particularly advantageous for detecting the pH of vaginal fluids which are infrequently discharged and typically in small volumes.

The pH sensor 101 may apply either a potentiometric approach or a conductometric approach. In the potentiometric approach, at least one of the conductive threads 404 functions as a reference electrode and at least another of the conductive threads 404 functions as a working electrode. The control unit 116 detects the potential changes between the working electrode and the reference electrode when exposed to a biological liquid. The control unit 116 is further configured to determine the acidity of the biological liquid based on the potential. Following the conductometric approach, the control unit 116 imposes a test signal on the first conductive thread 404-1, and if the biological liquid is contacting both the first and second conductive threads, the control unit 116 measures the feedback signal in the second conductive thread 404-2 responsive to the test signal 404-1. Based on the voltage drop between the test signal and feedback signal, the control unit 116 is configured to measure the resistance of the polyaniline. The control unit 116 is further configured to determine the acidity of the biological liquid based on the resistance of the PANI.

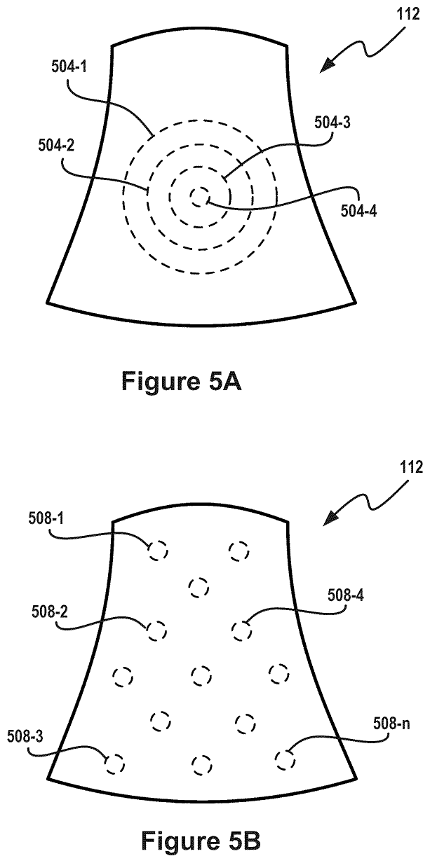

FIG. 5A shows another embodiment of the sensing element 112 in which the conductive threads 404 are arranged as a plurality of concentric circles. In FIG. 5A, the conductive threads 404 include a first concentric thread 504-1, a second concentric thread 504-2, a third concentric thread 504-3, and a fourth concentric thread 504-4, however the sensing element 112 may include any suitable number of concentric threads. In the embodiment shown in FIG. 5A, a single set of concentric circles is shown, however the sensing element 112 is not particularly limited. In other embodiments, the sensing element 112 may include multiple sets of concentric circles distributed over the textile.

FIG. 5B shows another embodiment of the sensing element 112 in which the conductive threads 404 are arranged as a plurality of scattered dots. In FIG. 5B, the conductive threads 404 include a first conductive thread 508-1, a second conductive thread 508-2, a third conductive thread 508-3, a fourth conductive thread 508-4, and an n^{th} conductive thread 508-n.

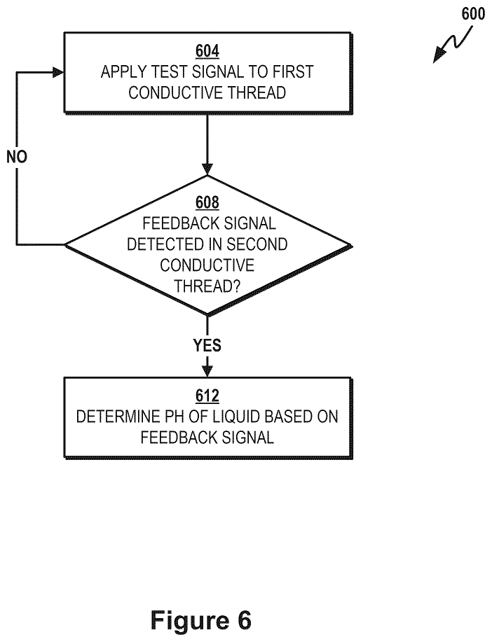

FIG. 6 is a block diagram of a method 600 for determining the acidity a biological liquid using the sensing element 112 according to one embodiment employing the conductometric approach. The method 600 will be explained with reference to the pH sensor 101 of FIG. 1 and the sensing element 112 of FIG. 4A, however the method 600 may be similarly applied to the sensing element 112 of FIGS. 4B, 5A, 5B, or any other suitable sensing element.

Block 604 comprises applying a test signal to at least a first conductive thread. In the pH sensor 101, block 604 is performed by the control unit control unit 116 which applies a test signal to at least the first conductive thread. In the embodiment shown in FIG. 4A, the control unit control unit 116 may apply the test signal to the first conductive thread 404-1, however, the order of the conductive threads 404 is not particularly limited to the arrangement shown in FIG. 4A, and in other examples, the first conductive thread 404-1 may be arranged between the second conductive thread 404-2 and the third conductive threads 404-3. Other arrangements are contemplated.

In examples where the control unit 116 applies the test signal to a plurality of the conductive threads 404, the control unit 116 may apply the test signal to alternating conductive threads 404. In other examples, the control unit 116 may apply the test signal to every third conductive thread 404. In further examples, the control unit 116 may apply the test signal to every fourth conductive thread 404. In the example shown in FIG. 4A, the control unit 116 may apply the test signal to the first conductive thread 404-1 and the third conductive thread 404-3.

The test signal comprises an electrical current. The electrical current may be between about 0.01 mA and about 0.1 mA, although the test signal is not particularly limited.

Block 608 comprises determining whether a feedback signal is detected in at least a second conductive thread 404-2. In the wearable device 100, block 608 is performed by the control unit 116 which determines whether a feedback signal has been detected in the second conductive thread 604-2, the feedback signal responsive to the test signal.

If no biological liquid is deposited on the textile to connect the first conductive thread 404-1 and the second conductive thread 404-2, the control unit 116 determines that no biological liquid is present on the textile, as shown at block 607, and the method 600 returns to block 604. Generally, data recorded when no biological liquid is present at the first time is disregarded or relevant data records are deleted from memory. Block 604 may be repeated continuously or periodically. In examples where the test signal is applied periodically, the frequency at which the test signal is applied may be between about 1 second and about 60 minutes. In some examples, the test signal is applied every 10 seconds. In some examples, the test signal is applied every 30 seconds. In some examples, the test signal is applied every 60 seconds. In some examples, the test signal is applied every 2 minutes. In some examples, the test signal is applied every 10 minutes. In some examples, the test signal is applied every 20 minutes. In some examples, the test signal is applied every 30 minutes. In some examples, the test signal is applied every 40 minutes. In some examples, the test signal is applied every 50 minutes. In some examples, the test signal is applied every hour.

If the biological liquid is deposited on the textile such that the biological liquid contacts first conductive thread 404-1 and the second conductive thread 404-2, forming a conductive bridge therebetween, the control unit 116 detects a feedback signal in the second conductive thread 404-2, and the method 600 proceeds to block 612. The first feedback signal will be detected in the second conductive threads 404-2 as long as the biological liquid contacts both the first and second conductive threads 404-1, 404-2 somewhere along the respective lengths and electrically connects the two conductive threads 404-1, 404-2.

In some examples, block 608 includes detecting the feedback signal in a plurality of the conductive threads 404.

At part of block 608, the control unit 116 may record in memory data representing the feedback signal. In some examples, block 608 includes measuring one or more electrical properties of the feedback signal. The one or more electrical properties of the feedback signal may be stored in memory. In preferred embodiments, the electrical property of the feedback signal is voltage since voltage is generally unaffected by contact between the biological liquid and the wearer's skin.

In some examples, block 608 includes recording the time of the feedback signal. In these examples, the control unit 116 includes a clock configured to record time. When the test signal is applied to the first conductive thread 404, the control unit 116 may retrieve the time from the clock and store the time in memory. As part of this step, the control unit 116 may further retrieve the data and store the date in memory.

The biological liquid is not particularly limited and may include sebum, sweat, vaginal discharge, cervical mucus, urine, blood, amniotic fluid, lochia, or the like.

Block 612 comprises determining the pH of the biological liquid based on the feedback signal. In the wearable device 100, the block 612 is performed by the processor 204 which computes the pH of the biological liquid based on the test signal applied at block 604 and the feedback signal received at block 608. As part of the comparison, the processor 204 determines the voltage drop between the test signal and the feedback signal. Based on the change in voltage, the processor 204 determines the resistance of the polyaniline.

The processor 204 determines the resistance of the polyaniline based on the test signal and the feedback signal and computes the pH of the biological liquid based on the resistance. The higher the potential difference, the lower the pH and vice versa. The processor 204 may compute a pH value based on the potential.

The pH value may be expressed as a numerical value between 0 and 14. In some examples, the control unit 116 converts the pH value to a classification such as very acidic, acidic, neutral, basic, or very basic.

In examples including a plurality of working electrodes and a plurality of reference electrodes, the control unit 116 may receive a plurality of feedback signals. In these examples, the processor 204 may be configured to compute a statistical value representing the pH of the biological liquid, the computation based on the plurality of feedback signals.

In the fertility monitoring system 300 of FIG. 3, the control unit 116 may transmit the pH to the computing device 338. In some examples, the control unit 116 controls the computing device 338 to display the pH value.

As a further part of method 600, the control unit 116, computing device 338 or the fertility tracking engine 312 may characterize the biological liquid based on the pH computed at block 612. The characterization may be based on a comparison between the pH and the reference data, which may be retrieved from memory. The characterization may be further based on user-generated data input at the computing device 338. Based on similarities between the reference data and the pH, the characterization may include identifying the biological liquid. In specific, non-limiting examples, the biological liquid may be identified as sebum, sweat, vaginal discharge, cervical mucus, urine, blood, amniotic fluid, lochia, the like, or a combination thereof.

The method 600 may further include identifying a reproductive status of the user. The reproductive status may represent a particular day or phase in the user's reproductive cycle. The reproductive status may be determined based on the characterization of the biological liquid, including the pH and identified type of liquid. The reproductive status may be further determined based on the reference data, the user-generated data, and other sensor data. Generally, the pH of vaginal discharge changes over the course of a reproductive cycle, and therefore the pH is indicative of the user's reproductive status. In addition to identifying the user's reproductive status, the method 600 may comprise determining a disease condition such as endometriosis, uterine fibroids, gynecologic cancer, polycystic ovary syndrome, congenital adrenal hyperplasia, sexually transmitted diseases, and the like.

The reproductive status determined in method 600 may be output at a display associated with the computing device 338.

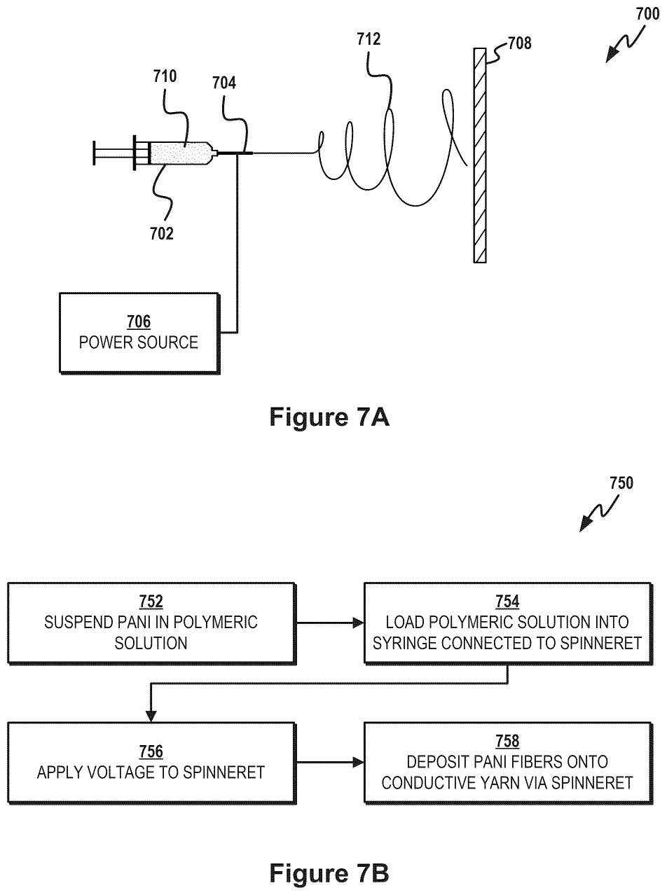

FIG. 7A shows an electrospinning apparatus 700 for manufacturing conductive threads. The electrospinning apparatus 700 includes a syringe 702, a spinneret 704, a power source 706, and a collector body 708. The syringe 702 is configured to receive a polymeric solution 710 which is ejected from the spinneret 704 to form fibers 712 that are deposited onto the collector body 708. The power source 706 is configured to apply a voltage to the spinneret 704 to induce an oscillating motion in the spinneret 704.

FIG. 7B shows a method 750 of manufacturing a pH sensor using electrospinning, according to one embodiment. In FIG. 7B, the method 750 is performed using the electrospinning apparatus 700 of FIG. 7A. The method 750 will be described herein with respect to the pH sensor 101 of FIG. 1.

At block 752, polyaniline (PANI) is suspended in a polymeric solution. The polymeric solution may comprise any such solution suitable for electrospinning. The particle size of the polyaniline may be optimized for suspension in the polymeric solution.

At block 754, the polymeric solution comprising the polyaniline is loaded into a syringe connected to a spinneret.

At block 756, a voltage is applied to the spinneret to induce an electrospinning effect.

At block 758, polyaniline fibers are deposited onto the conductive thread 404 to form a fibrous polyaniline coating. Block 758 comprises ejecting the polymeric solution from the syringe via the spinneret. Due to the whipping motion of the spinneret induced at block 756, the polyaniline forms elongated fibers which are deposited onto the conductive thread 404 to form the fibrous polyaniline coating thereupon. In some examples, the polyaniline forms a fibrous mat comprising the fibrous polyaniline coating.

The conductive thread 404 is positioned to receive the polyaniline fibers as they are ejected from the spinneret. The conductive thread 404 may be electrically grounded during the performance of block 758 or the conductive thread 404 may be positioned on a collection body which is grounded.

As part of method 750, characteristics of the polyaniline fibers may be optimized by controlling the voltage applied at block 756, spinneret diameter, feed rate, and distance to the conductive threads or collection body.

As a further part of method 750, two of the conductive threads 404 may be stitched into a textile. The conductive threads 404 may be stitched into the textile before or after the textile has been integrated into a garment to be worn by a user. During the stitching, the two conductive threads 404 may be arranged to form an interdigitated electrode. As a yet further part of method 750, the two conductive threads 404 may be connected to the control unit 116 which is configured to measure the pH of a biological liquid contacting the two conductive threads 404.

In view of the above, it will now be apparent that variant, combinations, and subsets of the foregoing embodiments are contemplated. For example, while the wearable device 100 has been described with respective to fertility monitoring, a skilled person will understand that the device and method can be similarly applied to other applications such as cancer detection, monitoring and detecting infectious diseases such as bacterial vaginosis, menopause monitoring, fitness monitoring, wellness, athletic training and performance, sleep tracking, and the like. Additionally, while the manufacturing method for the conductive threads is described above with respect to electrospinning, the polyaniline coating may instead be applied to the conductive thread as a film using electrochemical deposition.

It will now be apparent to a person of skill in the art that the present specification affords certain advantages over the prior art. The methods and devices described in this specification provide several advantages over existing textile-based sensing technologies. Electrospinning polyaniline directly onto conductive threads results in a fibrous, high-surface-area coating that enhances both the sensitivity and sensibility of the pH sensor. This structure maintains electrical performance while allowing the threads to remain flexible, breathable, and mechanically robust, supporting seamless integration into garments without compromising wearer comfort.

The use of conductive threads as the base for electrode formation enables washability and repeatability, overcoming common durability limitations in prior art systems. Unlike film-based or rigid sensors, the electrospun polyaniline coating can better withstand mechanical strain and washing cycles, making the sensor suitable for daily wear and reuse. The fibrous coating has a higher surface area promoting better interaction with the biological liquid which enhances responsiveness and reduces equilibration time. The fibrous polyaniline also allows the textile to maintain its air permeability, contributing to user comfort during extended use.

The flexible polyaniline-coated threads can be embroidered onto a textile or directly onto a completed garment, which supports rapid modification and high reproducibility. This allows consistent manufacturing at scale, while enabling customization for specific sensing requirements.

By electrically coupling the coated threads to a control unit and, in some embodiments, to a computing device, the sensor provides real-time pH detection of biological liquids. When used in a fertility monitoring system, this configuration supports automated, personalized health tracking based on reliable, reproducible signal interpretation.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A pH sensor for characterizing biological liquids, the pH sensor comprising:
a plurality of conductive threads disposed on a textile, including at least a first conductive thread and a second conductive thread spaced from the first conductive thread, the plurality of conductive threads including a fibrous polyaniline coating; and
a control unit electrically connected to the plurality of conductive threads, the control unit configured to:
apply a test signal to the first conductive thread,
record a feedback signal in the second conductive thread responsive to a biological liquid electrically connecting the first and second conductive threads;
compare the test signal to the feedback signal; and
determine a pH of the biological liquid based on the comparison between the test signal and the feedback signal.

2. The pH sensor of claim 1 wherein the plurality of conductive threads are selected from a group consisting of silver, copper, gold, aluminum, iron, steel, brass, graphite, conductive polymers, carbon nanotubes, and alloys thereof.

3. The pH sensor of claim 2 wherein the fibrous polyaniline coating is electrospun.

4. The pH sensor of claim 3 wherein the plurality of conductive threads are stitched into the textile.

5. The pH sensor of claim 4, wherein the plurality of conductive threads are arranged on the textile as interdigitated electrodes.

6. The pH sensor of claim 4, wherein the plurality of conductive threads are arranged substantially parallelly on the textile.

7. The pH sensor of claim 1, wherein the control unit is further configured to:
detect whether the feedback signal is transmitted by the second conductive thread; and
if the feedback signal is not transmitted, determine that no biological liquid is present on the textile.

8. A method of manufacturing a pH sensor, the method comprising:
suspending polyaniline in a polymeric solution;
loading the polymeric solution including the polyaniline into a syringe connected to a spinneret;
applying a voltage to the spinneret to induce an electrospinning effect; and
ejecting the polymeric solution through the spinneret to form polyaniline fibers and deposit the polyaniline fibers onto a conductive thread to form a fibrous polyaniline coating thereupon.

9. The method of claim 8 further comprising controlling one or more parameters selected from voltage, spinneret diameter, solution feed rate, and distance to the conductive thread to optimize a characteristic of the fibrous polyaniline coating.

10. The method of claim 9 further comprising selecting a particle size of the polyaniline to optimize the suspension.

11. The method of claim 10 wherein the conductive threads are selected from a group consisting of silver, copper, gold, aluminum, iron, steel, brass, graphite, conductive polymers, carbon nanotubes, and alloys thereof.

12. The method of claim 11 further comprising:
stitching two of the conductive threads into a textile; and
connecting the two conductive threads to a control unit configured to measure the pH of a biological liquid contacting the two conductive threads.

13. The method of claim 12 wherein stitching the two conductive threads into the textile further comprises arranging the two conductive threads onto the textile to form an interdigitated electrode.

14. A method of manufacturing a pH sensor comprising:
suspending polyaniline in a polymeric solution comprising 1 to 10% of the polyaniline and 10 to 20% of a polymer matrix; and
depositing a film comprising the polyaniline onto a conductive thread.

15. The method of claim 14 wherein the depositing the polyaniline film onto the conductive thread is performed by drop casting or doctor blading.

16. The method of claim 15 further comprising selecting a particle size of the polyaniline to optimize the suspension.

17. The method of claim 16 wherein the conductive threads are selected from a group consisting of silver, copper, gold, aluminum, iron, steel, brass, graphite, conductive polymers, carbon nanotubes, and alloys thereof.

18. The method of claim 17 further comprising:
stitching two of the conductive threads into a textile; and
connecting the two conductive threads to a control unit configured to measure the pH of a biological liquid contacting the two conductive threads.

19. The method of claim 18 wherein stitching the two conductive threads into the textile further comprises arranging the two conductive threads onto the textile to form an interdigitated electrode.
